(19) United States
(12) Patent Application Publication
(10) Pub. No.: US 2012/0296142 A1
(43) Pub. Date: Nov. 22, 2012

(54) ADDITION OF BASIC NITROGEN TO ALKYLATION REACTIONS

(75) Inventors: James R. Butler, Spicewood, TX (US); Sivadinarayana Chinta, Missouri City, TX (US)

(73) Assignee: FINA TECHNOLOGY, INC., Houston, TX (US)

(21) Appl. No.: 13/457,512

(22) Filed: Apr. 27, 2012

Related U.S. Application Data
(60) Provisional application No. 61/488,785, filed on May 22, 2011.

(51) Int. Cl. C07C 2/88; B01J 29/076; B01J 37/00; B01J 29/04

(52) U.S. Cl. 585/437; 502/87; 502/73; 502/74

(57) ABSTRACT

A process for making styrene including providing toluene, a co-feed, and a C₁ source to a reactor containing a catalyst having a total number of acid sites and reacting toluene with the C₁ source in the presence of the catalyst and the co-feed to form a product stream containing ethylbenzene and styrene where the co-feed removes at least a portion of the total number of acid sites on the catalyst. The co-feed can be selected from the group of ammonia, primary amines, and secondary amines, and combinations thereof. The C₁ source can be selected from methanol, formaldehyde, formalin, trioxane, methylformcel, paraformaldehyde, methylal, dimethyl ether, and combinations thereof.

### CROSS-REFERENCE TO RELATED APPLICATIONS

This application claims priority to U.S. Provisional Patent No. 61/488,785 filed on May 22, 2011.

### FIELD

The present invention generally relates to processes and catalysts used in hydrocarbon reactions, such as alkylation reactions. More specifically, the present invention relates to processes and catalysts for the alkylation reactions of toluene with a carbon source, such as methanol and/or formaldehyde, to produce styrene.

### BACKGROUND

A zeolite is a crystalline alumino-silicate that is well known for its utility in several applications. Zeolites have been used in dealkylation, transalkylation, isomerization, cracking, disproportionation, and dewaxing processes, among others. Its well-ordered structure is composed of tetrahedral AlO₄⁻⁴ and SiO₄⁻⁴ molecules bound by oxygen atoms that form a system of pores typically on the order of 3 Å to 10 Å in diameter. These pores create a high internal surface area and allow the zeolite to selectively adsorb certain molecules while excluding others, based on the shape and size of the molecules. Thus, a zeolite can be categorized as a molecular sieve. A zeolite can also be termed a “shape selective catalyst.” The small pores of the zeolite can restrict reactions to certain transition states or certain products, preventing shapes that do not fit the contours or dimensions of the pores.

The pores of a zeolite are generally occupied by water molecules and cations. Cations balance out the negative charge caused by trivalent aluminum cations that are coordinated tetrahedrally by oxygen anions. A zeolite can exchange its native cations for other cations; one example is the exchange of sodium ions for ammonium ions.

One alkylation reaction for which zeolite can be used as a solid basic catalyst is the alkylation of toluene with methanol and/or formaldehyde (ATM) to form styrene. Styrene, also known as vinyl benzene, is an organic compound having the chemical formula C₆H₅CHCH₂. The monomer styrene may be polymerized to form the polymer polystyrene. Polystyrene is a plastic that can form many useful products, including molded products and foamed products, all of which increase the need for production of styrene.

In the production of styrene, zeolite catalysts may be utilized. The zeolite used in the production of styrene can be categorized as a heterogeneous basic catalyst. The zeolite is characterized as heterogeneous because it is in a different phase than the reactants. The zeolite catalyst is solid and usually bound by an alumina or silica binder to increase to form a catalyst particle of the required size.

During the side chain alkylation of toluene with methanol to form styrene, water is released as a product of the reaction. Each water molecule includes two free electron pairs, wherein the free electron pairs of the water molecules may interact with the zeolite catalyst utilized in the alkylation process. The interaction of the free electron pairs of the water molecules and the zeolite catalyst can form additional acid sites on the zeolitic catalyst.

Bulk zeolitic catalysts typically contain an abundance of acid sites. In the presence of alkylation reactions, however, these acid sites may contribute to the production of unwanted by-products, such as xylenes.

Therefore, it would be desirable to reduce the amount of the acid sites on a zeolitic catalyst used in the production of styrene. It would also be desirable to use an alkylation catalyst capable of increasing the selectivity to styrene.

### SUMMARY

The present invention in its many embodiments relates to a process of making styrene. In an embodiment of the present invention, a process is provided for making styrene including reacting toluene with a C₁ source in the presence of a catalyst and a co-feed in a reactor to form a product stream including ethylbenzene and styrene. The catalyst includes a total number of acid sites. The co-feed is selected from the group of ammonia, primary amines, and secondary amines, and combinations thereof, and the co-feed removes at least a portion of the total number of acid sites on the catalyst. The C₁ source is selected from the group of methanol, formaldehyde, formalin, trioxane, methylformcel, paraformaldehyde, methylal, dimethyl ether, and combinations thereof. The toluene conversion can be at least 10%. The selectivity to styrene can be at least 40%.

In an embodiment, either by itself or in combination with any other embodiment, the catalyst includes at least one promoter on a support material. The promoter can be selected from the group of Co, Mn, Ti, Zr, V, Nb, K, Cs, Ga, B, P, Rb, Ag, Na, Cu, Mg, Fe, Mo, Ce, or combinations thereof. The support material can include a zeolite.

In an embodiment, either by itself or in combination with any other embodiment, the co-feed adds basic sites to the catalyst. The co-feed can inhibit the reactivity of at least a portion of the total number of acid sites on the catalyst by the molecules of the co-feed occupying spatial volume near the acid sites of the catalyst. The co-feed can be added to the catalyst prior to the toluene and the C₁ source. Optionally, the co-feed is simultaneously fed to the reactor with the toluene and the C₁ source. The co-feed can be present in the reactor in a co-feed to toluene and C₁ source of at least 0.01 wt %. Optionally, the co-feed is present in amounts of 0.01 to 5.0 wt % of the total feed stream.

The various embodiments of the present invention can be joined in combination with other embodiments of the invention and the listed embodiments herein are not meant to limit the invention. All combinations of various embodiments of the invention are enabled, even if not given in a particular example herein.

### BRIEF DESCRIPTION OF DRAWINGS

FIG. 1 illustrates a flow chart for the production of styrene by the reaction of formaldehyde and toluene in the presence of a co-feed, wherein the formaldehyde is first produced in a separate reactor by either the dehydrogenation or oxidation of methanol and is then reacted with toluene to produce styrene.

FIG. 2 illustrates a flow chart for the production of styrene by the reaction of formaldehyde and toluene in the presence of a co-feed, wherein methanol and toluene are fed into a reactor, wherein the methanol is converted to formaldehyde and the formaldehyde is reacted with toluene to produce styrene.

### DETAILED DESCRIPTION

The present invention relates to increasing the selectivity in an alkylation process, for example an alkylation of toluene with methanol (ATM) process. More specifically, the present invention is related to the modification of a catalyst, such as a zeolite catalyst, to reduce the number of acid sites on the catalyst. The catalyst is modified by the addition of a molecule having a more basic character than that of water or alcohol in a way that reduces the total number of acid sites of the zeolite catalyst, such that by-product formation is inhibited and styrene selectivity is increased. Also, the present invention includes the addition of a molecule having a steric character that would allow the molecule to occupy spatial volume near the acidic sites of the zeolite.

The present catalyst is adaptable to use in the various physical forms in which catalysts are commonly used. The catalyst of the invention may be used as a particulate material in a contact bed or as a coating material on structures having a high surface area. If desired, the catalyst can be deposited with various catalyst binder and/or support materials.

A catalyst comprising a substrate that supports a promoting metal or a combination of metals can be used to catalyze the reaction of hydrocarbons. The method of preparing the catalyst, pretreatment of the catalyst, and reaction conditions can influence the conversion, selectivity, and yield of the reactions.

The various elements that make up the catalyst can be derived from any suitable source, such as in their elemental form, or in compounds or coordination complexes of an organic or inorganic nature, such as carbonates, oxides, hydroxides, nitrates, acetates, chlorides, phosphates, sulfides and sulfonates. The elements and/or compounds can be prepared by any suitable method, known in the art, for the preparation of such materials.

In accordance with an embodiment of the current invention, toluene is reacted with a carbon source capable of coupling with toluene, which can be referred to as a C₁ source, to produce styrene and ethylbenzene. In an embodiment, the C₁ source includes methanol or formaldehyde or a mixture of the two. In an alternative embodiment, toluene is reacted with one or more of the following: formalin, trioxane, methylformcel, paraformaldehyde and methylal. In a further embodiment, the C₁ source is selected from the group of methanol, formaldehyde, formalin (37-50% H₂CO in solution of water and MeOH), trioxane (1,3,5-trioxane), methylformcel (55% H₂CO in methanol), paraformaldehyde and methylal (dimethoxymethane), dimethyl ether, and combinations thereof. In an embodiment the C₁ source can include formaldehyde synthesized insitu in a separate reactor using methanol as feed.

Formaldehyde can be produced either by the oxidation or dehydrogenation of methanol.

In an embodiment, formaldehyde is produced by the dehydrogenation of methanol to produce formaldehyde and hydrogen gas. This reaction step produces a dry formaldehyde stream that may be preferred, as it would not require the separation of the water prior to the reaction of the formaldehyde with toluene. The dehydrogenation process is described in the equation below:

CH₃OH→CH₂O+H₂

Formaldehyde can also be produced by the oxidation of methanol to produce formaldehyde and water. The oxidation of methanol is described in the equation below:

2 CH₃OH+O₂→2 CH₂O+2 H₂O

In the case of using a separate process to obtain formaldehyde, a separation unit may then be used in order to separate the formaldehyde from the hydrogen gas or water from the formaldehyde and unreacted methanol prior to reacting the formaldehyde with toluene for the production of styrene. This separation would inhibit the hydrogenation of the formaldehyde back to methanol. Purified formaldehyde could then be sent to a styrene reactor and the unreacted methanol could be recycled.

Although the reaction has a 1:1 molar ratio of toluene and the C₁ source, the ratio of the C₁ source and toluene feedstreams is not limited within the present invention and can vary depending on operating conditions and the efficiency of the reaction system. If excess toluene or C₁ source is fed to the reaction zone, the unreacted portion can be subsequently separated and recycled back into the process. In one embodiment the ratio of toluene:C₁ source can range from between 100:1 to 1:100. In alternate embodiments the ratio of toluene:C₁ source can range from 50:1 to 1:50; from 20:1 to 1:20; from 10:1 to 1:10; from 5:1 to 1:5; from 2:1 to 1:2.

In an embodiment, the reactants, toluene and the C₁ source, are combined with a co-feed having basic properties. In an embodiment, the co-feed is selected from the group of ammonia, primary amines, and secondary amines, and combinations thereof. In an alternate embodiment, the co-feed comprises amines. The co-feed may be combined with the reactants in any desired amounts. In an embodiment, the process of the present invention contains a co-feed of from 0.01 to 5.0 wt % with respect to the feed. In another embodiment, the process of the present invention contains a co-feed of from 0.1 to 3.0 wt % with respect to the feed. In an embodiment, the co-feed is added in amounts of from 0.1 to 1.0 wt % with respect to the feed.

In an embodiment, the co-feed comprises amines. In an alternate embodiment, the co-feed is selected from the group of ammonia, primary amines, and secondary amines, and combinations thereof. Nonlimiting examples of primary amines include methylamine, ethylamine, aniline and the like. Nonlimiting examples of secondary amines include methylethanolamine, dimethylamine, pyrrolidine, diethylamine, N-methylaniline and the like. Nonlimiting examples of other suitable amines include pyrrole, pyridine, and the like.

The addition of a support material to improve the catalyst physical properties is possible within the present invention. Binder material, extrusion aids or other additives can be added to the catalyst composition or the final catalyst composition can be added to a structured material that provides a support structure. For example, the final catalyst composition can include an alumina or aluminate framework as a binder. Upon calcination these elements can be altered, such as through oxidation which would increase the relative content of oxygen within the final catalyst structure. The combination of the catalyst of the present invention combined with additional elements such as a binder, extrusion aid, structured material, or other additives, and their respective calcination products, are included within the scope of the invention.

The powder form of a zeolite and other catalysts may be unsuitable for use in a reactor, due to a lack of mechanical stability, making alkylation and other desired reactions difficult. To render a catalyst suitable for the reactor, it can be combined with a binder to form an aggregate, such as a zeolite aggregate. The binder-modified zeolite, such as a zeolite aggregate, will have enhanced mechanical stability and strength over a zeolite that is not combined with a binder, or otherwise in powder form. The aggregate can then be shaped or extruded into a form suitable for the reaction bed. The binder can desirably withstand temperature and mechanical stress and ideally does not interfere with the reactants adsorbing to the catalyst. In fact, it is possible for the binder to form macropores, much greater in size than the pores of the catalyst, which provide improved diffusional access of the reactants to the catalyst.

Binder materials that are suitable for the present invention include, but are not limited to, silica, alumina, titania, zirconia, zinc oxide, magnesia, boria, silica-alumina, silica-magnesia, chromia-alumina, alumina-boria, silica-zirconia, silica gel, clays, kaolin, montmorillonite, modified clays, similar species, and any combinations thereof. The most frequently used binders are amorphous silica and alumina, including gamma-, eta-, and theta-alumina. It should be noted that a binder can be used with many different catalysts, including various forms of zeolite and non-zeolite catalysts that require mechanical integrity inside a reactor.

As used herein, the term “metal ion” is meant to include all active metal ions and similar species, such as metal oxides, nanoparticles, and mixed metal oxide phases, capable of being added to a binder and enabling the binder to reduce the acidity, or increase the basicity or basic strength, of the supported catalyst without adversely affecting the catalyst that it supports or causing significant by-product formation at reaction conditions.

The metal ion can be added to the zeolite, or non-zeolite, in the amount of 0.1% to 50%, optionally 0.1% to 20%, optionally 0.1% to 5%, by weight of the zeolite, or non-zeolite. The metal ion can be added to the zeolite, or non-zeolite, by any means known in the art. Generally, the method used is incipient wetness impregnation, wherein the metal ion precursor is added to an aqueous solution, which solution is poured over a zeolite. After sitting for a specified period, the zeolite is dried and calcined, such that the water is removed with the metal ion deposited in the pores of the zeolite. The ion-modified zeolite can then be mixed with a binder, or another catalyst, by any means known in the art. The mixture is shaped via extrusion or some other method into a form such as a pellet, tablet, cylinder, cloverleaf, dumbbell, symmetrical and asymmetrical polylobates, sphere, or any other shape suitable for the reaction bed. The shaped form is then usually dried and calcined. Drying can take place at a temperature of from 100° C. to 200° C. Calcining can take place at a temperature of from 400° C. to 900° C. in a substantially dry environment.

For the present invention, the catalyst can be a zeolite, but can also be a non-zeolite. Zeolite materials suitable for this invention may include silicate-based zeolites and amorphous compounds such as faujasites, mordenites, etc. Silicate-based zeolites are made of alternating SiO₄ and MO₄ tetrahedra, where M is an element selected from the Groups 1 through 16 of the Periodic Table (new IUPAC). These types of zeolites have 4-, 6-, 8-, 10-, or 12-membered oxygen ring channels. An example of zeolites of this invention can include faujasites. Other suitable zeolite and zeolite-like catalysts include the types zeolite A, zeolite X, zeolite Y, zeolite L, zeolite beta, ZSM-5, MCM-22, MCM-41, as well as faujasite, mordenite, chabazite, offretite, clinoptilolite, erionite, sihealite, and the like. It is possible to generate crystals that are not alumino-silicates but behave similarly to zeolite, including aluminophosphates (ALPO) and silicoaluminophosphates (SAPO).

Another method of altering a zeolite is by ion-exchange. For example, the hydrogen form of a zeolite can be produced by ion-exchanging beta zeolite with ammonium ions. Metal ions can also be incorporated into a zeolite, either by ion-exchange or another method. Further, the silica/alumina ratio of the zeolite can be altered, via a variety of methods, such as dealumination by steaming or acid washing to increase the silica/alumina ratio. Increasing the amount of silica relative to alumina can have the effect of increasing the catalyst hydrophobicity. The silica/alumina ratio can range from less than 0.5 to 500 or greater. In an embodiment, the zeolite materials suitable for this invention are characterized by silica to alumina ratio (Si/Al) of less than 1.5. In another embodiment, the zeolite materials are characterized by a Si/Al ratio ranging from 1.0 to 200, optionally from 1.0 to 100, optionally from 1.0 to 50, optionally from 1.0 to 10, optionally from 1.0 to 2.0, optionally from 1.0 to 1.5. Some catalysts other than zeolitic catalysts can also be used with a binder of the present invention, including catalysts that fall into the general categories of molecular sieves and/or solid acid catalysts.

A variety of zeolites and non-zeolites are available for use in the present invention. The various catalysts listed in this disclosure are not meant to be an exhaustive list, but is meant to indicate the type of catalysts for which may be useful in the present invention.

In one embodiment, the catalyst can be prepared by combining a substrate with at least one promoter element. Embodiments of a substrate can be a molecular sieve, from either natural or synthetic sources. Zeolites and zeolite-like materials can be an effective substrate. Alternate molecular sieves also contemplated are zeolite-like materials such as the crystalline silicoaluminophosphates (SAPO) and the aluminophosphates (ALPO).

Upon contact with the co-feed, at least a portion of the total number of acid sites on the zeolite may be selectively poisoned or masked by the co-feed. Also some of the oxygen in the zeolite lattice can be replaced by nitrogen that may be present in the co-feed. In an embodiment, the co-feed may have a more basic character than that of water produced in the alkylation of toluene with methanol. The alkylation of toluene with methanol includes alcohol, wherein the co-feed can have a more basic character than the alcohol. In an embodiment, the co-feed may have a steric character that may allow at least a portion of the co-feed to occupy spatial volume near the acid sites of the zeolite. In a further embodiment, the addition of the co-feed may alter the structural dimensions of the catalyst, resulting in the catalyst having an altered shape selectivity.

In a conventional process of the alkylation of toluene with methanol, a product stream can be produced including water and alcohol. The alcohol and/or water can interact with at least a portion of the zeolite, wherein the portion includes at least one zeolite site, thereby converting the zeolite site to an acid site. Such a conversion increases the total number of acid sites and increases the acidity of the zeolite catalyst. Optionally, water and/or alcohol can interact with at least one acid site of the total number of acid sites, thereby increasing the acidity of the acid site and correspondingly increasing the acidity of the zeolite catalyst.

In an embodiment of the present invention, the alkylation of toluene with methanol occurs in the presence of a co-feed including amines. The amines are of a greater basic character than the water and alcohol produced by the alkylation of toluene with methanol. The chemistry of amines is dominated by the lone pair of electrons on nitrogen. Because of this lone pair, amines are both basic and nucleophilic. Amines can react with acids to form acid-base salts.

Accordingly, the co-feed including amines can interact with at least the portion of the zeolite including at least one zeolite site, thereby prohibiting water or alcohol from interacting with the zeolite site. Optionally, the amines may interact with at least one acid site of the total number of acid sites, thereby neutralizing the acid site. Thus, the prohibition of the interaction of water and/or alcohol with the zeolite site by the addition of the amines can result in the reduction of acidity of the zeolite catalyst and an increase in basicity of the zeolite catalyst.

In an embodiment, the co-feed including amines can have a steric character that may allow at least a portion of the co-feed to occupy spatial volume near the acid sites of the zeolite. The steric character of the co-feed allows at least a portion of the amines to occupy spatial volume near the acid sites of the zeolite, thereby blocking water and alcohol from reaching the acid sites. The blockage of these sites by the addition of amines may increase the basicity of the zeolite catalyst.

An improvement in side chain alkylation selectivity may be achieved by treating a molecular sieve zeolite catalyst with chemical compounds to inhibit the external acidic sites and to minimize aromatic alkylation on the ring positions. Another means of improvement of side chain alkylation selectivity can be to impose restrictions on the catalyst structure to facilitate side chain alkylation. In one embodiment the catalyst used in an embodiment of the present invention is a basic or neutral catalyst.

The catalytic reaction systems suitable for this invention can include one or more of the zeolite or amorphous materials modified for side chain alkylation selectivity. A non-limiting example can be a zeolite promoted with one or more of the following: Co, Mn, Ti, Zr, V, Nb, K, Cs, Ga, B, P, Rb, Ag, Na, Cu, Mg, Fe, Mo, Ce, or combinations thereof. In an embodiment, the zeolite can be promoted with one or more of Ce, Cu, P, Cs, B, Co, Ga, or combinations thereof. In an embodiment the promoter exchanges with Na within the zeolite. The promoter can also be attached to the zeolite material in an occluded manner. In an embodiment the amount of promoter is determined by the amount needed to yield less than 0.5 mol % of ring alkylated products such as xylenes from a coupling reaction of toluene and a C₁ source.

In an embodiment, the catalyst contains greater than 0.1 wt % of at least one promoter based on the total weight of the catalyst. In another embodiment, the catalyst contains up to 5 wt % of at least one promoter. In a further embodiment, the catalyst contains from 1 to 3 wt % of at least one promoter.

The operating conditions of the reactors and separators will be system specific and can vary depending on the feedstream composition and the composition of the product streams. The reactor for the reactions of methanol to formaldehyde and toluene with formaldehyde will operate at elevated temperatures and may contain a basic or neutral catalyst system. The temperature can range in a non-limiting example from 250° C. to 750° C., optionally from 300° C. to 500° C., optionally from 375° C. to 450° C. The pressure can range in a non-limiting example from 0.1 atm to 70 atm, optionally from 0.1 atm to 35 atm, optionally from 0.1 atm to 10 atm, optionally from 0.1 atm to 5 atm.

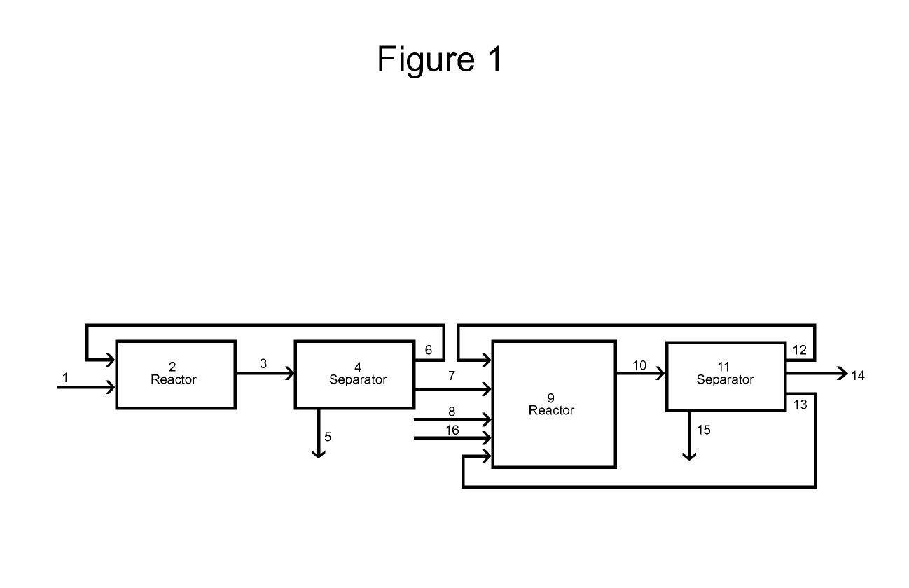

In FIG. 1 there is a simplified flow chart of one embodiment of the styrene production process described above. In this embodiment, a first reactor (2) is either a dehydrogenation reactor or an oxidation reactor. This reactor is designed to convert at least part of the first methanol feed (1) into formaldehyde. The gas product (3) of the reactor is then sent to alkylation reactor with or without membrane treatment to remove hydrogen.

In one embodiment the first reactor (2) is a dehydrogenation reactor that produces formaldehyde and hydrogen and the separation unit (4) is a membrane capable of removing hydrogen from the product stream (3).

In an alternate embodiment the first reactor (2) is an oxidative reactor that produces product stream (3) comprising formaldehyde and water. The product stream (3) comprising formaldehyde and water can then be sent to the second reactor (9) without a separation unit (4).

The formaldehyde feed stream (7) is then reacted with a feed stream of toluene (8) and a co-feed stream (16) in a second reactor (9). In the embodiment illustrated in FIG. 1, the co-feed stream (16) includes amines. The toluene and formaldehyde and methanol react in the presence of the co-feed to produce styrene. The product (10) of the second reactor (9) may then be sent to an optional separation unit (11) where any unwanted byproducts (15) such as water can separated from the styrene, unreacted formaldehyde or methanol and unreacted toluene. Any unreacted methanol, formaldehyde (12) and a mixture (13) of any unreacted toluene and unreacted amines can be recycled back into the reactor (9). A styrene product stream (14) can be removed from the separation unit (11) and subjected to further treatment or processing if desired.

The operating conditions of the reactors and separators will be system specific and can vary depending on the feedstream composition and the composition of the product streams. The reactor (9) for the reaction of toluene and formaldehyde will operate at elevated temperatures and may contain a basic or neutral catalyst system. The temperature can range in a non-limiting example from 250° C. to 750° C., optionally from 300° C. to 500° C., optionally from 375° C. to 450° C. The pressure can range in a non-limiting example from 0.1 atm to 70 atm, optionally from 0.1 atm to 35 atm, optionally from 0.1 atm to 10 atm, optionally from 0.1 atm to 5 atm.

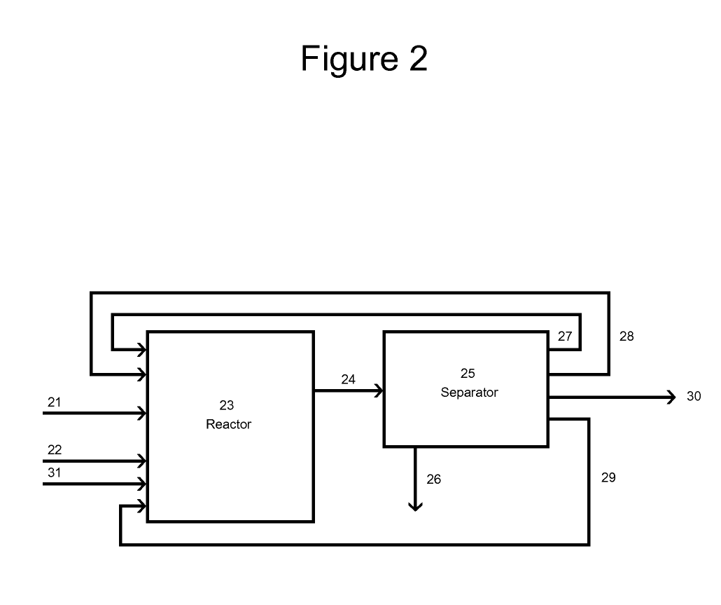

FIG. 2 is a simplified flow chart of another embodiment of the styrene process discussed above. A C₁ source containing feed stream (21) is fed along with a feed stream of toluene (22) and a co-feed stream (31) in a reactor (23). In the embodiment illustrated in FIG. 2, the co-feed stream (31) includes amines. Toluene and the C₁ source then react in the presence of the co-feed to produce styrene. The product (24) of the reactor (23) may then be sent to an optional separation unit (25) where any unwanted byproducts (26) can be separated from the styrene, and any unreacted C₁ source, unreacted methanol, unreacted formaldehyde, unreacted amines and unreacted toluene. Any unreacted methanol (27), unreacted formaldehyde (28) and a mixture (29) of any unreacted toluene and unreacted amines can be recycled back into the reactor (23). A styrene product stream (30) can be removed from the separation unit (25) and subjected to further treatment or processing if desired.

The operating conditions of the reactors and separators will be system specific and can vary depending on the feedstream composition and the composition of the product streams. The reactor (23) for the reactions of methanol to formaldehyde and toluene with formaldehyde will operate at elevated temperatures and may contain a basic or neutral catalyst system. The temperature can range in a non-limiting example from 250° C. to 750° C., optionally from 300° C. to 500° C., optionally from 375° C. to 450° C. The pressure can range in a non-limiting example from 0.1 atm to 70 atm, optionally from 0.1 atm to 35 atm, optionally from 0.1 atm to 10 atm, optionally from 0.1 atm to 5 atm.

In the embodiments illustrated in the Figures, the co-feed includes amines having a boiling point substantially similar to the boiling point of the toluene in the toluene feed stream. Amines with this characteristic are selected so that any unreacted toluene and unreacted amines may be separated together, or co-distilled, whereby a mixture of the unreacted toluene and unreacted amines may be recycled back to the reactor. Choosing amines having boiling point properties substantially similar to the toluene in the toluene feed stream can be economically beneficial in that further separation components do not have to be added or constructed to separate the amines from the product stream.

In an alternate embodiment, the co-feed includes amines having boiling point properties separate and distinct from the boiling point properties of the toluene in the toluene feed streams such that any unreacted co-feed is separated from the product stream independently of any unreacted toluene. The order of separation from the product stream can depend on the separation unit and/or physical properties, e.g., boiling points, of the co-feed and toluene.

Upon deactivation, the zeolite may require a regeneration procedure to be performed. Some methods of regenerating a zeolite include heating to remove adsorbed materials, ion exchanging with cesium to remove unwanted cations One solution involves flushing the catalyst with benzene. Other solutions generally involve processing the catalyst at high temperatures using regeneration gas and oxygen. According to one procedure, a zeolite beta can be regenerated by heating the catalyst first to a temperature in excess of 300° C. in an oxygen-free environment. Then an oxidative regeneration gas can be supplied to the catalyst bed with oxidation of a portion of a relatively porous coke component to produce an exotherm moving through the catalyst bed. Either the temperature or the oxygen content of the gas can be progressively increased to oxidize a porous component of the coke. Again, regeneration gas can be supplied, wherein the gas has either increased oxygen content or increased temperature to oxidize a less porous refractory component of the coke. The regeneration process can be completed by passing an inert gas through the catalyst bed at a reduced temperature.

In one embodiment, the present invention is for an alkylation process containing a catalyst, wherein toluene, a C₁ source, and a co-feed are fed to a reactor containing the catalyst wherein the co-feed removes at least a portion of the total number of acid sites on the catalyst. In another embodiment, the present invention is for an alkylation process containing a catalyst, wherein toluene, a C₁ source, and a co-feed are fed to a reactor containing the catalyst wherein the co-feed adds basic sites to the catalyst. In yet another embodiment, the present invention is for an alkylation process containing a catalyst, wherein toluene, a C₁ source, and a co-feed are fed to a reactor containing the catalyst wherein the molecules of the co-feed can occupy spatial volume near the acidic sites of the zeolite.

The term “molecular sieve” refers to a material having a fixed, open-network structure, usually crystalline, that may be used to separate hydrocarbons or other mixtures by selective occlusion of one or more of the constituents, or may be used as a catalyst in a catalytic conversion process.

Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

The term “regenerated catalyst” refers to a catalyst that has regained enough activity to be efficient in a specified process. Such efficiency is determined by individual process parameters.

The term “regeneration” refers to a process for renewing catalyst activity and/or making a catalyst reusable after its activity has reached an unacceptable/inefficient level. Examples of such regeneration may include passing steam over a catalyst bed or burning off carbon residue, for example.

The term “zeolite” refers to a molecular sieve containing a silicate lattice, usually in association with some aluminum, boron, gallium, iron, and/or titanium, for example. In the following discussion and throughout this disclosure, the terms molecular sieve and zeolite will be used more or less interchangeably. One skilled in the art will recognize that the teachings relating to zeolites are also applicable to the more general class of materials called molecular sieves.

The various embodiments of the present invention can be joined in combination with other embodiments of the invention and the listed embodiments herein are not meant to limit the invention. All combinations of various embodiments of the invention are enabled, even if not given in a particular example herein.

While illustrative embodiments have been depicted and described, modifications thereof can be made by one skilled in the art without departing from the spirit and scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.).

Depending on the context, all references herein to the “invention” may in some cases refer to certain specific embodiments only. In other cases it may refer to subject matter recited in one or more, but not necessarily all, of the claims. While the foregoing is directed to embodiments, versions and examples of the present invention, which are included to enable a person of ordinary skill in the art to make and use the inventions when the information in this patent is combined with available information and technology, the inventions are not limited to only these particular embodiments, versions and examples. Also, it is within the scope of this disclosure that the embodiments disclosed herein are usable and combinable with every other embodiment disclosed herein, and consequently, this disclosure is enabling for any and all combinations of the embodiments disclosed herein. Other and further embodiments, versions and examples of the invention may be devised without departing from the basic scope thereof and the scope thereof is determined by the claims that follow.

What is claimed is:

1. A process for making styrene comprising:
reacting toluene with a C₁ source in the presence of a catalyst and a co-feed in a reactor to form a product stream comprising ethylbenzene and styrene; wherein
the C₁ source is selected from the group consisting of methanol, formaldehyde, formalin, trioxane, methylformcel, paraformaldehyde, methylal, dimethyl ether, and combinations thereof;
the catalyst comprises a total number of acid sites; and
the co-feed is selected from the group consisting of ammonia, primary amines, and secondary amines, and combinations thereof, and the co-feed removes at least a portion of the total number of acid sites on the catalyst.

2. The process of claim 1, wherein the catalyst comprises at least one promoter on a support material.

3. The process of claim 2, wherein the at least one promoter is selected from the group consisting of Co, Mn, Ti, Zr, V, Nb, K, Cs, Ga, B, P, Rb, Ag, Na, Cu, Mg, Fe, Mo, Ce, and combinations thereof.

4. The process of claim 2, wherein the support material comprises a zeolite.

5. The process of claim 1, wherein the co-feed adds basic sites to the catalyst.

6. The process of claim 1, wherein the co-feed inhibits the reactivity of at least a portion of the total number of acid sites on the catalyst by the molecules of the co-feed occupying spatial volume near the acid sites of the catalyst.

7. The process of claim 1, wherein the co-feed is added to the catalyst prior to the toluene and the C₁ source.

8. The process of claim 1, wherein the co-feed is simultaneously fed to the reactor with the toluene and the C₁ source.

9. The process of claim 1, wherein reacting toluene with the C₁ source in the presence of the catalyst and the co-feed further forms water, wherein the basicity of the co-feed is greater than the basicity of water, whereby the co-feed removes at least a portion of the total number of acid sites on the catalyst by the interaction of the co-feed with the portion of the total number of acid sites on the catalyst such that the water is unable to interact with the portion of the total number of acid sites on the catalyst.

10. The process of claim 1, wherein co-feed is present in the reactor in a co-feed to toluene and C₁ source of at least 0.01 wt %.

11. The process of claim 1, wherein the co-feed is present in amounts of 0.01 to 5.0 wt % of the total feed stream.

12. The process of claim 1 wherein the reaction has a toluene conversion of at least 10%.

13. The process of claim 1 wherein the reaction has a toluene selectivity to styrene of at least 40%.

14. A method of preparing a catalyst, the method comprising:
contacting a substrate with a first solution comprising at least one promoter; and
contacting a catalyst disposed in an alkylation reactor with a co-feed selected from the group consisting of ammonia, primary amines, and secondary amines, and combinations thereof;
wherein the catalyst comprises at least one promoter and has a total number of acid sites and the contacting of the substrate with the solution subjects the substrate to the addition of at least one promoter.

15. The method of claim 14, wherein the substrate is a zeolite.

16. The method of claim 14, wherein the at least one promoter is selected from the group consisting of Co, Mn, Ti, Zr, V, Nb, K, Cs, Ga, B, P, Rb, Ag, Na, Cu, Mg, Fe, Mo, Ce, and combinations thereof.

17. The method of claim 14, wherein the co-feed removes at least a portion of the total number of acid sites on the catalyst.

18. The catalyst of claim 14, wherein spatial volume near the acid sites of the catalyst can be occupied by molecules of the co-feed.

19. The process of claim 14 wherein the reaction has a toluene conversion of at least 10%.

20. A process of producing styrene comprising:
reacting toluene with a C₁ source in the presence of a catalyst and a co-feed in a reactor to form a product stream comprising ethylbenzene, styrene, and water; wherein
the C₁ source is selected from the group consisting of methanol, formaldehyde, formalin, trioxane, methylformcel, paraformaldehyde, methylal, and combinations thereof;
the catalyst comprises a total number of acid sites;
the co-feed is selected from the group consisting of ammonia, primary amines, and secondary amines, and combinations thereof;
the molecules of the co-feed can occupy spatial volume near at least a portion of the total number of acid sites on the catalyst; and
the co-feed removes at least a portion of the total number of acid sites on the catalyst.
